(19) United States
(12) Patent Application Publication
(10) Pub. No.: US 2010/0119589 A1
(43) Pub. Date: May 13, 2010

(54) LIPOSOMAL COMPOSITION OF ANTIOXIDANTS FOR INHALATIONS CARRIED OUT DURING LUNG AND UPPER RESPIRATORY TRACT DISEASES

(75) Inventors: Alla Anatolievna SELISCHEVA, Moscow (RU); Vladimir Petrovich TIKHONOV, Moscow (RU)

Correspondence Address: FOLEY AND LARDNER LLP;SUITE 500, 3000 K STREET NW, WASHINGTON, DC, 20007, US

(73) Assignee: OTKRYTOE AKTSIONERNOE OBSCHESTVO ZAVOD EKOLOGICHES, Moscow (RU)

(21) Appl. No.: 12/375,852

(22) PCT Filed: Jul. 23, 2007
(86) PCT No.: PCT/RU2007/000403

§ 371 Date: Nov. 5, 2009

(30) Foreign Application Priority Data
Aug. 2, 2006 (RU) 2006128005

(51) Int. Cl. A61K 9/127; A61P 11/00; A61K 31/355

(52) U.S. Cl. 424/450; 514/458

(57) ABSTRACT

The invention relates to medicine and pharmacology, in particular to liposomal compositions of antioxidants suitable for inhalations in lung and upper respiratory tract diseases. The present composition of antioxidants for inhalation in lung and upper respiratory tract diseases represents an emulsion of phospholipids in form of liposomes with an average particle size of 0.2-0.4 μm, in the membrane of which the flavonoid dihydroquercetin and wheat germ oil are introduced. The latter contains the hydrophobic antioxidants tocopherols (TP) (vitamin E) and carotenoids. The aqueous phase of the emulsion contains sodium chloride, the water-soluble antioxidants ascorbic acid (vitamin C), N-acetyl-L-cysteine and sodium benzoate. The composition of antioxidants is selected in order to provide the possibility of administering the individual vitamins in minimum doses, and to ensure that the content of active, not oxidized form of the antioxidants is not reduced during storage.

### TECHNICAL FIELD

The invention relates to medicine and pharmacology, in particular to the production of liposomal compositions that can be used in lung and upper respiratory tract diseases.

PRIOR ART

Usually the peroxidic oxidation of phospholipids of biomembranes occurs slowly, as there are several systems in the tissues protecting from oxidation, the systems regulating the oxidation process. One of these systems are low molecular oxidation inhibitors, namely, antioxidants. In the lungs ascorbic acid, uric acid, lipoic acid, glutathione and its precursor cysteine serve as water-soluble anti-oxidants, whereas vitamin E, vitamin A and its provitamin carotene are present as hydrophobic antioxidants [B. C. Schock, I. S. Young, V. Browb, P. S. Fitch, M. D. Shields, M. Ennis, Antioxidant and oxidative stress in BAL fluid of atopic asthmatic children, Pediatr. Res. 2003.53(3); 375-381].

In the development of various pathologies the antioxidant protection proves not to be sufficient, and the peroxidic oxidation of phospholipids proceeds much faster than normally. This phenomenon is called oxidative stress, and it is associated with diseases like chronical cardiovascular diseases (atherosclerosis, cardio ischemia, hypertension); obliterating diseases of peripheric arteries; diseases of the venous system (hemorrhoids, varicosis and others); arthritis; depressions; prevention of tumor diseases. An acceleration of the oxidation is noted during aging of an organism, in smokers and in the case of intoxications. Studies have shown that local oxidative stress develops in the lungs of smokers being the basis for development of pretumor and neoplastic processes in the organs of the respiratory system.

Some tissues (of the brain, the retina, the lungs) are more sensitive to oxidative stress. This is due to their particular chemical structure and metabolism. Oxidative stress is believed to play a key role in the development of pulmonary injury, where the concentration of low molecular antioxidants in the lungs decreases and the quantity of products of peroxidic oxidation increases, in particular decomposition products of parts of the surfactant, predominantly, of phospholipids and proteins. Therefore, nowadays an antioxidant therapy is prescribed along with chemotherapy in the treatment of lung diseases (M. Christofidou-Solomidou, V. R. Muzykantov, Antioxidant strategies in respiratory medicine, Treat Respir Med. 2006; 5(1): 47-78).

However, the development of oxidative stress is only one of the main factors in pathological conditions that reflects an imbalance in the system “activated oxygen metabolites-antioxidants”.

In aerobic organisms continuously reactions proceed in which so-called activated oxygen metabolites are involved. There is numerous evidence of the important role of the latter in the evolution of inflammatory and destructive processes, vasodepression and decreased vascular permeability, in the activation of the peroxidic oxidation of lipids (POL) of the cell membranes, the development of atherosclerosis, the immune response, cell proliferation, exacerbation of infections and others.

Therefore application of individual known correctors of oxidative stress as, for example, vitamin E, vitamin C, or dibunol, does not suffice, but also toxical manifestations of activated oxygen metabolites must be neutralized.

Furthermore, still another problem must be resolved in the development of efficient preparations for the treatment of lung diseases, which is associated with the extremely low bioavailability of vitamins that serve as antioxidants, a fact that research has confirmed over the last years. For example, the bioavailability of vitamin E in tablets was as low as 3 percent [Scott W. Leonard, C. K. Good, E. T. Gugger, M. G. Traber, Vitamin E bioavailability from fortified breakfast cereal is greater than that from encapsulated supplements^{1,2,3}, Amer. J. Clin. Nutrition, vol. 79, no. 1, 86-92, 2004]. In this regard it must be considered that only a small part of these 3 percent reaches the lungs with the blood stream.

Inhalation are used to ensure a more complete delivery of drugs or pathogenetic agents to the lungs and to the affected part of them [Russian patent application no. 93003456]. But only by means of an ultrasonic inhaler or pneumo-inhaler particles of less than one micron of size can be obtained, which reach the lower sections of the lungs while larger drops remain in the upper part of the respiratory tract [N. A. Geppe, Nebulayzernaya terapiya pri obostrenii bronkhialnoy astmy u detej (“Inhalation therapy in case of aggravation of bronchial asthma in children”), Russkiy Meditsinskiy Zhurnal vol. 7, no. 11, 1999, p. 505]. In normal oil-in-water emulsions, into which hydrophobic antioxidants can be introduced, the fat drops are of a size of more than 1 μm, and they must be stabilized by adding emulsifiers. On the other hand, when liposomes of phospholipids are obtained by means of a special technology, they originally are particles of less than 1 μm of size.

Liposomal compositions represent one of the most prospective forms for use in pulmonology.

Earlier the therapeutical effect has been demonstrated that is reached by inhalation through an ultrasonic inhaler or pneumo-inhaler by using
liposomes of phospholipids of the egg for the treatment of bronchial asthma and obstructive bronchitis [K. A. Masuev, E. A. Limarenko, A. G. Tchutchalin, Pulmonologiya (“Pulmonology”), 1991, vol. 3, p. 68-69];
a mixture of phospholipids from bovine lungs [Russian patent no. 2149016] for the treatment of the distress syndrome.

Known are liposomal compositions for the treatment of tuberculosis and pyoinflammatory diseases, comprising rifampicine, phospholipids, ascorbic acid, a-tocopherol, and an isotonic solution of sodium chloride (Russian patent no. 2217128).

Said technical solution solves a restricted problem, namely, it provides efficient delivery of a certain agent, i.e., rifampicine, and prevents its decomposition.

Also compositions with two antioxidants have been developed (P.N. Shek, Z. E. Suntres, J. I. Brooks, Liposomes in pulmonary applications: physicochemical considerations, pulmonary distribution and antioxidant delivery, J. Drug Target, 1994; 2(5): 431-42, PMID: 7704488 [PubMed—indexed for MEDLINE]).

A liposomal composition for inhibition of free radicals that suitable for use inter alia in damage of lung tissue, comprising at least two antioxidants selected from beta-carotene, vitamin E, ascorbic acid, glutathione, niacin, and additionally at least one metal, such as Zn, Se, Cr, Cu, Mn, and natural or synthetic phospholipids that are suitable for generating liposomes, or a mixture of such phospholipids (U.S. Pat. No. 6,764,693) may be qualified as the technical solution closest to the present invention. However, said composition is not sufficiently efficient.

Furthermore, the possibility of occurrence of negative effects is known (G. W. Comstock, A. J. Alberg, H. Y. Huang, K. N. Wu, A. E. Burke, S. C. Hoffmann, E. P. Norkus, M. Gross, R. G. Cutler, J. S. Morris, V. L. Spate and K. J. Helzlsouer, “The risk of developing lung cancer associated with antioxidants in the blood: Ascorbic acid, carotenoids, α-tocopherol, selenium, and total peroxyl radical absorbing capacity”, Cancer Epidemiol. Biomarkers Prey. 6: 907-916, 1997).

### DISCLOSURE OF THE INVENTION

Object of the present invention is to develop a composition of an emulsion of phospholipids for inhalative administration, in which the particles (liposomes) are less than one micron of size also in case of long-time storage and do not oxidize due to the presence of antioxidants of various types. The composition of antioxidants is selected such as to enable administration of individual vitamins in minimum doses, ensuring that the content of the active, not oxidized form of the antioxidants is not reduced during storage.

For achieving said object it is proposed:
1. to use a mixture of phospholipids for obtaining the emulsion;
2. to obtain liposomes of 0.2-0.4 μm of size from the emulsion by means of extrusion in the presence of 0.9 percent sodium chloride, which ensures physical stability of the liposomes;
3. to use wheat germ oil as the source of hydrophobic antioxidants, representing tocopherols and carotenoids;
4. to introduce an antioxidant of the class of flavones into the preparation, namely, dihydroquercetin;
5. to introduce the hydrophobic antioxidants into the liposomes in the initial phase, before obtaining the liposomes by extrusion;
6. to ensure regeneration of the active form of the hydrophobic antioxidants by introducing water-soluble antioxidants into the composition (ascorbic acid and N-acetylcysteine);
7. to use inhalative administration of the preparation by means of a individual ultrasonic inhaler or a individual pneumo-inhaler.

All proposals listed above aim at an effective preparation with low doses of antioxidants, the latter not showing any side effects (damage of the membrane structure) or negative effects (promotion of oxidation).

The biologically active compound that is enclosed in the liposomes is protected from the action of enzymes. This increases the effectiveness of the preparations that are likely to decompose in biological liquids. The compounds are released from the liposomes gradually, and thus a prolonged action is achieved. A further advantage of the liposomal form of preparations as compared to traditional preparations consists in the capacity of the liposomes to interact with phagocytes and to activate them (Biomembrany, 2000). In this way the local immunity is activated. Lastly, liposomes are absolutely non-toxic because they consist of phospholipids that represent natural biodegradable compounds.

Thus, the object of the present invention is a liposomal composition of antioxidants for inhalation in lung and upper respiratory tract diseases, the composition representing an emulsion of phospholipids in form of liposomes with an average particle size of 0.2-0.4 μm, in the membrane of which the flavonoid dihydroquercetin and wheat germ oil are introduced, wherein the aqueous phase of the emulsion contains sodium chloride and the water-soluble antioxidants ascorbic acid (vitamin C), N-acetyl-L-cysteine, and sodium benzoate, with the following ratio of the components (percent by weight):

| | |
|---|---|
| phospholipids | 1-20 |
| dihydroquercetin | 0.1-1.1 |
| wheat germ oil, containing hydrophobic | 0.1-1.1 |
| antioxidants tocopherols (TP) (vitamin E) | |
| and carotenoids | |
| ascorbic acid | 0.04-0.06 |
| N-acetyl-L-cysteine | 0.1-1.1 |
| sodium benzoate | 0.12-0.2 |
| 0.9 percent sodium cloride solution, | rest |
| at pH = 6.4 ± 0.5. | |

Composition of antioxidants. The use of several antioxidants in one preparation has important advantages, but also disadvantages. The advantages consist in that some antioxidants, e.g., tocopherol and ascorbic acid, can mutually potentiate their activity. At the same time, however, a mutual weakening of effectiveness is possible, depending on the interaction of the antioxidants. This has been shown in model systems, for example, for tocopherol and carotenoids [E. B. Burlakova, N. M. Storozhok, N. G. Khrapova, Izutcheniye additivnogo antiokislitelnogo deystviya summy prirodnykh antioksidantov lipidov (“Study of the additive antioxidative effect of a totality of natural lipids as antioxidants”), Voprosy medicinskoy khimii 1990, vol. 36, 4th edition, p. 72-74].

Wheat germ oil is known to be a source of various antioxidants. According to evidence in the literature the following ingredients have been found in oils of wheat germ:
saturated fatty acids: tetradecanoic acid, palmitic acid, stearic acid, erucic acid, gondonic acid;
mono- and polyunsaturated fatty acids: oleinic acid, linolenoic acid, arachidonic acid;
liposoluble vitamins: tocopherols, carotenoids, ergocalciferol, with a tocopherol content of up to 1360 mg per cent;
water-soluble vitamins: folic acid (vitamin B9), pantothenic acid.

Furthermore, lecithin, methionine, and phytosterols have been found in the composition of this oil.

Wheat germ oil is so valuable because it contains various tocopherols and carotenoids, these antioxidants being present in a ratio that ensures not only the antioxidative effect of each of these ingredients, but even potentiates this effect. The highest amount of up to 945 mg percent is of a-type of tocopherol. However, in the process of interaction with free radicals a-tocopherol oxidizes to form a α-tocopheryl racidal, and in the course of a number of reactions its antioxidative activity decreases. Unlike α-tocopherol β- and δ-tocopherols show less anti-radical activity, but their antioxidative activity is higher.

The introduction of hydrophobic antioxidants into the membrane of liposomes strengthens their antioxidative activity.

In connection with liposomes the bioavailability of the tocopherols is significantly better.

Requirement of regeneration of the active form of the antioxidants.

Antioxidants belonging to the class of polynuclear phenols (dihydroquercetin and tocopherols comprised in wheat germ oil) interrupt the peroxidic oxidation of lipids by reacting with the peroxidic radicals and formation of phenoxy radicals. Thus concentration of active form of the antioxidants in the preparation decreases, but the resulting oxidized form turned out to be toxic. On the other hand it is undesirable to use high concentrations of liposoluble antioxidants, for example, tocopherol, since the latter can disturbe the structure of the liposomes if it reaches certain concentrations [R. P. Evstigeeva, I. M. Volkov, V. V. Tchudinova, Biol. Membrany, 1998, vol. 15, no. 2, p. 119-136].

In order to maintain the concentration of active form of liposoluble antioxidants, water-soluble antioxidants (ascorbic acid and N-acetylcysteine) are added to the formulation regenerating the radicals to the original compounds. The content of ascorbic acid and N-acetylcysteine (N-acetyl-L-cysteine) is much higher than the concentration of polynuclear phenols, and, therefore, the active form of the latter is maintained for a long time. In living organisms dehydroascorbat that forms as a result of oxidation of ascorbic acid is reduced to ascorbic acid.

N-Acetyl-L-cysteine is a modified form of the amino acid cysteine, and it is a known mucolytic agent. A significant advantage of acetylcysteine is its antioxidative activity. N-Acetylcysteine is a precursor of one of the most important components of antioxidative protection, namely, of glutathione which has a protecting function in the respiratory system and inhibits the damaging effect of oxidants. Said property is especially important for aged patients where oxidation processes are significantly stronger and the antioxidative effect of the blood plasma is weaker. However, up till now in the case of separate administration, fairly high doses of 600-1000 mg are required, and this could cause undesirable side effects, related in particular to an effect on the blood vessels.

The composition is formulated in a way that on the one hand the mere antioxidant activity of each single component develops, and on the other hand the active form of all antioxidants is maintained.

### BEST EMBODIMENT OF THE INVENTION

Below the chemical structure of the antioxidants which are comprised in the formulation of the preparation is described, and results of studies are presented that support the claimed antioxidative capacity of the composition.

The combination of several types of water-soluble and liposoluble antioxidants in a formulation can have an unexpected result, e.g., it can lead to an increased peroxidic oxidation of lipids. This is associated with the possibility of interaction between the antioxidants being either synergistic, i.e., they mutually enhance their effect, or agonistic, when they mutually inhibit the effect.

The composition according to the invention represents the most effective mixtures, that is, the mixtures that are most capable of inhibiting peroxidic oxidation of the phospholipids in the form of liposomes.

The invention can be illustrated by the following examples:

### Example of Preparation of a Liposomal Composition (Preparation “Pulmo-aktiv”) for Inhalation:

1. Preparation of the Hydrophobic Phase

1.1 25 g of a dry mixture of natural or synthetic phospholipids are fed into a glass of a volume of 2 ml. The main component of the mixture must be phosphotidyicholine (PCh). A small amount of phosphatidylethanolamine (PE), phosphatidylinosite (PI) and phosphatidylserine (PS) is acceptable. The concentration of neutral phospholipids and fatty acids must not exceed 5 percent, and the concentration of lyso-components must not exceed 1 percent.

In the present example a mixture of soybean phospholipids of the company Lipoid (Germany) is used, types S-73 and S-45, in a ratio of 1:3, i.e., 6,25 g S-75 and 18,75 g 5-45.

1.2. 25 g ethyl alcohol are added.

1.3. 1 g wheat germ oil is added to the mixture of phospholipids and alcohol.

The glass is placed in a shaking apparatus for 2-4 min. in order to mix the oil with the alcohol and the phospholipids.

1.4. An alcoholic solution of dihydroquercetin is prepared by pouring 5 g alcohol into a glass of a volume of 20 ml containing 1 g dihydroquercetin, and a solution is obtained by stirring slowly at 50° C.

1.5. The alcoholic dihydroquercetin solution is added to the mixture obtained according to 1.3. Then the glass is placed in a shaking apparatus for mixing the components.

2. Preparation of the Aqueous Phase

2.1. 0.5 g ascorbic acid, 1.0 g N-acetyl-L-cysteine and 1.5 g sodium benzoate are solved in 500 g of a 0.9 per cent sodium chloride solution.

2.2. The pH value of the solution is measured. Then 1 g 0.1 M sodium chloride is added to the aqueous solution. Then the pH value is measured again; it should be 6.4±0.5.

3. Preparation of a Rough Emulsion

3.1. The aqueous solution obtained according to 2.2. is added to the mixture obtained according to 1.5. Afterwards homogenisation is effected in a high speed turbomixer.

3.2. 300 g of 0.9 per cent sodium cloride are added to the emulsion obtained.

3.3. The pH value of the emulsion is measured; it should be 6.4±0.5. If the pH value is correct, i.e., 6.4±0.5, 0.9 per cent sodium chloride is added up to 1000 g.

4. Preparation of the Liposomes

4.1. The rough emulsion obtained according to 3.3. is placed in the chamber of a homogenizer of the type “Donor” (Russia) or “a-Laval” (Sweden), and 4-12 treatment cycles are performed under a pressure of (5.4-6.5)·107 Pa.

4.2. The pH value of the solution is measured; it should be 6.4±0.5.

5. Determination of Particle Size

5.1. The size of the liposomes in the preparation “Pulmo-aktiv” obtained according to 4.1. is determined by dynamic light diffusion method with the help of an Autosizer 111 “Malvern” (England).

5.2. 7 ml of the preparation “Pulmo-aktiv” are placed in the container of the individual ultrasonic inhaler “Ingport” (“lsomed”, Russia) and treated with ultrasound for 20 min. Afterwards the particle size is determined again.

The antioxidative activity of preparations with varying combinations of the ingredients was evaluated on the basis of their effect on the increase of concentration of POL products (malondialdehyde or products that are sentitive to thiobarbituric acid (TBA)) in an aqueous dispersion of soybean phospholipids. For this evaluation the concentration in the soybean phospatidylcholine liposomes was measured at an initial moment and after incubation at 37° C. for 60 min. in the absence and presence of POL starters, namely, Fe³⁺ ions and ascorbate. The results are shown in the table.

Originally a 2.0 percent aqueous dispersion of phospholipides with antioxidants, where wheat germ oil is is also present, contains an insignificant amount of products sensitive to TBA (0.8 nmol/ml). This amount rises to 2-2.5 times as much as a result of heating for 60 min. When starters are added to the original system the concentration of POL products rises to 40 nmol/ml during incubation at 37° C. for 60 min., i.e., will be 80 times as high.

As can be seen from the data in the table, an amount of 5 mg/ml of wheat germ oil significantly inhibits the POL, and if the concentration is raised 4 times the POL inhibition is practically complete. The minimum effective concentration of dihydroquercetin has been proved to be 0.03 mg/ml. However, when wheat germ oil was added a surprising effect occurred: the antioxidant activity of dihydroquercentin decreased. In other words, an antagonistic relationship was observed between antioxidants that are contained in wheat germ oil, and dihydroquercetin.

The minimum inhibiting concentration of ascorbic acid proved to be 2.5 mg/ml. However, when both wheat germ oil and ascorbic acid are simultaneously present, the concentration of the acid can be decreased by 5 times.

The addition of ascorbic acid to a mixture of wheat germ oil and dihydroquercetin ensured an effective inhibition even with minimal concentrations of the ingredients. Thus, only this last composition of antioxidants met the set requirements.

TABLE

| | wheat germ | dihydro- | ascorbic | products sensitive |
|---|---|---|---|---|
| | oil | quercetin | acid | to TBA |
| no. | (mg/ml) | (mg/ml) | (mg/ml) | (nmol/ml) |
| 1 | 0 | 0 | 0 | 40 |
| 2 | 5 | 0 | 0 | 7 |
| 3 | 20 | 0 | 0 | 1 |
| 4 | 0 | 0.03 | 0 | 1 |
| 5 | 0 | 0 | 2.5 | 4 |
| 6 | 1 | 0 | 0.25 | 10 |
| 7 | 5 | 0 | 0.5 | 1 |
| 8 | 5 | 0.03 | 0 | 3 |
| 9 | 5 | 0.03 | 0.25 | 2 |

Increase of the amount of products sensitive to TBA [nmol/ml] in multi lamellar vesicles of soybean phosphatidylcholine (PCh) containing N-acetyl-L-cysteine as a result of oxidation, the latter caused by introduction of an oxidation starter Pe3+/ascorbat (60 min, 37° C.) into the system, in the absence and presence of various antioxidants (AO).

### INDUSTRIAL APPLICABILITY

The use of the present invention for the treatment of diseases of the lungs and the upper respiratory tract ensures the effectiveness of the preparation that contains low doses of antioxidants that do not have any side effect or negative effect.

Furthermore, a biologically active compound that is included in liposomes is protected from the action of enzymes. This increases the effectiveness of the preparation, as the latter are likely to decompose in biological liquids. Thanks to the gradual release of the agents from the liposomes a long term effect is achieved.

1. Liposomal composition of antioxidants for inhalation in lung and upper respiratory tract diseases, characterized in that it represents an emulsion of natural and/or synthetic phospholipids in form of liposomes with an average particle size of 0.20.4 μm, in the membrane of which the flavonoid dihydroquercetin and wheat germ oil are introduced, wherein the aqueous phase of the emulsion contains sodium chloride in form of a 0.9 per cent solution and the water-soluble antioxidants ascorbic acid (vitamin C), N-acetyl-L-cysteine and sodium benzoate, with the following ratio of the components (percent by weight):

| | |
|---|---|
| phospholipids | 2.25-2.75 |
| dihydroquercetin | 0.9-1.1 |
| wheat germ oil comprising the hydrophobic | 0.9-1.1 |
| antioxidants tocopherols (TP) (vitamin E) | |
| and carotenoids | |
| ascorbic acid | 0.04-0.06 |
| N-acetyl-L-cysteine | 0.9-1.1 |
| sodium benzoate | 0.12-0.17 |
| 0.9 per cent sodium cloride solution | rest |
| at pH = 6.4 ± 0.5. | |
